SEQUENCE LISTING (8 sequences)

SEQ ID NO: 1 - DNA, 22 bp, synthetic construct
  source 1..22: mol_type = other DNA
tgcgtcagtgtgtctaccatgt

SEQ ID NO: 2 - DNA, 20 bp, synthetic construct
  source 1..20: mol_type = other DNA
agacatgtcctcctggaaca

SEQ ID NO: 3 - DNA, 22 bp, synthetic construct
  source 1..22: mol_type = other DNA
tgtgctggatttgttttttgtt

SEQ ID NO: 4 - DNA, 22 bp, synthetic construct
  source 1..22: mol_type = other DNA
gtgtcgtggaccatcagggcca

SEQ ID NO: 5 - DNA, 20 bp, synthetic construct
  source 1..20: mol_type = other DNA
acatgtgggggatgttcatt

SEQ ID NO: 6 - DNA, 20 bp, synthetic construct
  source 1..20: mol_type = other DNA
gaggccatgttacggttttg

SEQ ID NO: 7 - DNA, 24 bp, synthetic construct
  source 1..24: mol_type = other DNA
atcctgacggagcgcggttacagc

SEQ ID NO: 8 - DNA, 24 bp, synthetic construct
  source 1..24: mol_type = other DNA
tgcccatctcctgctcaaagtcca

(19) United States
(12) Patent Application Publication
(10) Pub. No.: US 2024/0167000 A1
(43) Pub. Date: May 23, 2024

(54) CONSTRUCTION METHOD AND APPLICATION OF OVARIAN GRANULOSA CELL LINE OF ONCORHYNCHUS MYKISS

(71) Applicant: Heilongjiang River Fishery Research Institute of Chinese Academy of Fishery Sciences, Harbin (CN)

(72) Inventors: Tianqing HUANG, Harbin (CN); Enhui LIU, Harbin (CN); Gefeng XU, Harbin (CN); Wei GU, Harbin (CN); Gaochao WANG, Harbin (CN); Bingqian WANG, Harbin (CN); Kaibo GE, Harbin (CN)

(21) Appl. No.: 18/513,069

(22) Filed: Nov. 17, 2023

(30) Foreign Application Priority Data
Nov. 18, 2022 (CN) 202211445569.X

(51) Int. Cl. C12N 5/09; C12N 5/00; C12Q 1/6851; C12Q 1/6886

(52) U.S. Cl. CPC C12N 5/0693; C12N 5/0081; C12Q 1/6851; C12Q 1/6886; C12N 2509/00; C12Q 1/6804; C12Q 2600/158

(57) ABSTRACT

A construction method and an application of an ovarian granulosa cell line of Oncorhynchus mykiss are provided. The construction method includes transferring a single follicle to a complete Minimum Essential Medium (MEM) containing collagenase H for digestion and transferring primary ovarian granulosa cells to 18° C. for constant temperature culture. The disclosure also provides an ovarian granulosa cell line constructed according to the construction method, and the application of the ovarian granulosa cell line in the separation and culture of fish granulosa cells, the molecular regulation mechanism of granulosa cells and the establishment of hormone metabolism cell models. The construction method of the disclosure has strong repeatability and simple operation steps, and after primary culture, the granulosa cells have good growth state and stable physiological state.

### CROSS-REFERENCE TO RELATED APPLICATIONS

This application claims priority of Chinese Patent Application No. 202211445569.X, filed on Nov. 18, 2022, the entire contents of which are incorporated herein by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: 347101PPH_14338-sequence
Creation date: Nov. 14, 2023
Byte size: 8,314

### TECHNICAL FIELD

The disclosure relates to the technical field of cells, and in particular to a construction method and an application of an ovarian granulosa cell line of Oncorhynchus mykiss.

### BACKGROUND

As an important part of follicles, an ovarian granulosa cell (GSC) regulates the development and atresia of follicles through gonadotropin receptors, steroid hormones and various growth factors, and plays an important role in the normal development of oocytes and follicles. Therefore, in mammals, the ovarian granulosa cell is often used as an in vitro research model for ovarian function research. However, there are few studies on granulosa cells in fish at present, causing the in vitro culture model of granulosa cells to be scarce.

According to the disclosure, the ovarian tissue of sexually mature female Oncorhynchus mykiss is taken as the research object, a primary ovarian granulosa cell culture system of Oncorhynchus mykiss is established, and the separated cells are identified, so as to provide reference materials for studying the molecular regulation mechanism of granulosa cells of Oncorhynchus mykiss and establishing hormone metabolism cell models in the future.

### SUMMARY

The objective of the present disclosure is to provide a construction method and an application of an ovarian granulosa cell line of Oncorhynchus mykiss, so as to solve the problems existing in the prior art. The ovarian granulosa cell line of Oncorhynchus mykiss provided by the present disclosure lays a theoretical foundation as well as scientific and technological support for subsequent scientific research on Oncorhynchus mykiss reproduction and cell aspects, and the provided cell line may be used to study the molecular mechanism of meiosis in the process of Oncorhynchus mykiss reproduction regulation.

In order to achieve the above objective, the present disclosure provides the following scheme.

The disclosure provides a construction method of an ovarian granulosa cell line of Oncorhynchus mykiss, which includes following steps:
(1) transferring a single follicle to a complete Minimum Essential Medium (MEM) containing collagenase H for digestion;
(2) putting a digested follicle in a culture dish containing Hanks' Balanced Salt Solution (HBSS), puncturing the digested follicle and incubating the digested follicle overnight to obtain a digestive solution containing follicle envelope, and treating and placing the digestive solution into the MEM for resuspension and precipitation to obtain primary ovarian granulosa cells;
(3) transferring the primary ovarian granulosa cells to 18° C. for constant temperature culture, and passaging once every 4-5 days when a cell adherence density reaches over 80%; and
(4) identifying the primary ovarian granulosa cells by an immunofluorescence method and a real-time fluorescence quantitative polymerase chain reaction (real-time PCR) method.

Optionally, in the S1, the single follicle is separated from an ovarian tissue washed by a tissue washing solution.

Optionally, in the S1, a concentration of the collagenase H is 0.4 international unit per milliliter (UI/mL).

Optionally, in the S1, a condition for the digestion includes a digestion at 18° C. for 3-4 hours (h).

Optionally, in the S2, components of the culture dish also include NaHCO₃ (sodium bicarbonate) and 1% bovine serum albumin.

Optionally, in the S2, the treating includes filtering the digestive solution with a 40 micrometer (μm) cell sieve, centrifuging at 1000 revolutions per minute (rpm) for 7 minutes (min), and discarding a supernatant; components of the MEM also include 10% fetal bovine serum and 1% Penicilllin-Streptomycin Solution.

Optionally, in the S4, the immunofluorescence method uses a granulosa cell marker gene follicle-stimulating hormone receptor (FSHR) as a marker to carry out immunofluorescence detection in granulosa cells; the real-time PCR method is based on expression levels of cyp19a1, fox12a and shbgb in ovarian granulosa cells.

The disclosure also provides an ovarian granulosa cell line constructed according to the construction method.

The disclosure also provides an application of the ovarian granulosa cell line in separation and culture of fish granulosa cells, a molecular regulation mechanism of granulosa cells and an establishment of hormone metabolism cell models.

The disclosure discloses the following technical effects.

According to the disclosure, ovarian tissue of female Oncorhynchus mykiss is obtained under an aseptic condition, and immediately put in a tissue washing solution for washing; after washing, the single follicle is separated and digested in the complete MEM containing collagenase H, then the follicle is punctured in HBSS and incubated overnight, followed by further filtering and purification by centrifugation; the complete MEM containing fetal bovine serum and Penicilllin-Streptomycin Solution (penicillin-streptomycin) is used for resuspension and precipitation, and the primary ovarian granulosa cells of Oncorhynchus mykiss are obtained.

According to the separation and primary culture method of ovarian granulosa cells of Oncorhynchus mykiss provided by the disclosure, the obtained granulosa cells have a stable growth state and are capable of continuously passaging, and the ovarian granulosa cells of Oncorhynchus mykiss may be obtained in batches through passaging.

The construction method of ovarian granulosa cells of Oncorhynchus mykiss is simple and convenient to operate and has strong repeatability, and may provide guidance for the separation and culture of granulosa cells of other fish.

The ovarian granulosa cell line of Oncorhynchus mykiss obtained by the disclosure may provide reference materials for the molecular regulation mechanism of granulosa cells of Oncorhynchus mykiss and the establishment of hormone metabolism cell models in the future.

### BRIEF DESCRIPTION OF THE DRAWINGS

In order to explain the embodiments of the present disclosure or the technical scheme in the prior art more clearly, the practical drawings needed in the embodiments are briefly introduced below. Obviously, the drawings described below are only some embodiments of the present disclosure, and other drawings may be obtained according to these drawings without creative work for ordinary people in the field.

FIG. 1A is a morphological diagram of ovarian granulosa cells of Oncorhynchus mykiss under a microscope with a magnification of 10×.

FIG. 1B is the morphological diagram of ovarian granulosa cells of Oncorhynchus mykiss under a microscope with a magnification of 20×.

FIG. 2A shows cells before antibody staining.

FIG. 2B shows stained cells.

FIG. 3A shows results of gene expression level investigation of a granulosa cell marker gene cyp19a1.

FIG. 3B shows results of gene expression level investigation of a granulosa cell marker gene fox12a.

FIG. 3C shows results of gene expression level investigation of a granulosa cell marker gene shbgb (sex hormone-binding globulinb).

FIG. 4A is a morphological diagram of cells incubated and digested in Minimum Essential Medium (MEM) containing collagenase H.

FIG. 4B is a morphological diagram of cells incubated and digested in MEM without collagenase H.

### DETAILED DESCRIPTION OF THE EMBODIMENTS

A number of exemplary embodiments of the present disclosure will now be described in detail, and this detailed description should not be considered as a limitation of the present disclosure, but should be understood as a more detailed description of certain aspects, characteristics and embodiments of the present disclosure.

It should be understood that the terminology described in the present disclosure is only for describing specific embodiments and is not used to limit the present disclosure. In addition, for the numerical range in the present disclosure, it should be understood that each intermediate value between the upper limit and the lower limit of the range is also specifically disclosed. The intermediate value within any stated value or stated range and every smaller range between any other stated value or intermediate value within the stated range are also included in the present disclosure. The upper and lower limits of these smaller ranges may be independently included or excluded from the range.

Unless otherwise specified, all technical and scientific terms used herein have the same meaning as commonly understood by one of ordinary skill in the art to which this disclosure relates. Although the present disclosure only describes the preferred methods and materials, any methods and materials similar or equivalent to those described herein can also be used in the practice or testing of the present disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes may be made to the specific embodiments of the present disclosure without departing from the scope or spirit of the present disclosure. Other embodiments will be apparent to the skilled person from the description of the disclosure. The specification and embodiments of this application are only exemplary.

The terms “comprising, “including”, “having” and “containing” used in this specification are all open terms, which means including but not limited to.

Unless otherwise specified, the reagents used in the embodiments of this application can be obtained from the general market.

### Embodiment 1

A construction method of an ovarian granulosa cell line of Oncorhynchus mykiss is as follows.

1. Separation and Primary Culture of Ovarian Granulosa Cells of Oncorhynchus mykiss

The sexually mature female Oncorhynchus mykiss (over 3-year old) is taken from the Bohai Sea Cold Water Fish Experimental Station (Mudanjiang) of Heilongjiang River Fishery Research Institute of Chinese Academy of Fishery Sciences, transported back to a laboratory with moisture, and is anesthetized to death with 100 mg/L 3-Aminobenzoic acid ethyl ester methanesulfonate (MS-222). After wiping the surface of the fish with alcohol cotton balls, the ovarian tissue is taken out with sterile scissors and put into sterile phosphate-buffered saline (PBS) (containing 1% penicillin and streptomycin, pH 7.4), and then brought into the sterile console in cell room.

The tissue is rinsed with PBS solution containing 1% Penicilllin-Streptomycin Solution in a sterile culture dish for 3 times to clean off attachments. After cutting open the outer membrane of the tissue with sterile tweezers, the single follicle is separated and then transferred to the complete Minimum Essential Medium (MEM, from Sigma) containing 0.4 international unit per milliliter (UI/mL) collagenase H for incubation and digestion for 3 hours (h), and the incubated follicle is transferred to a culture dish (containing Hank's Balanced Salt Solution (HBSS), 4.17 mM NaHCO₃ (sodium bicarbonate), and 1% bovine serum albumin), and the follicle is punctured using the tip of a 1 mL syringe, and then the follicle envelope is soaked in the HBSS overnight.

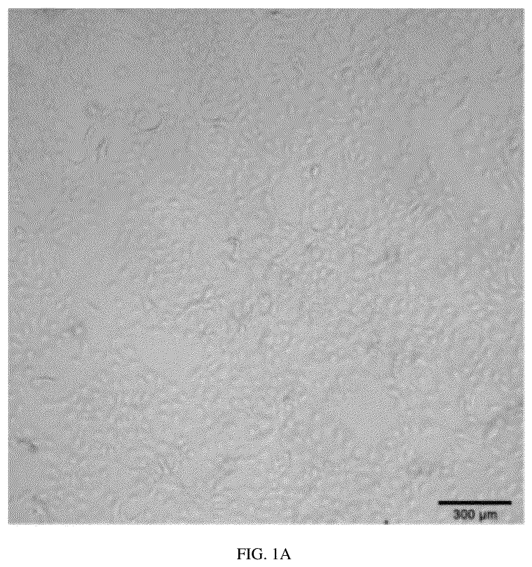

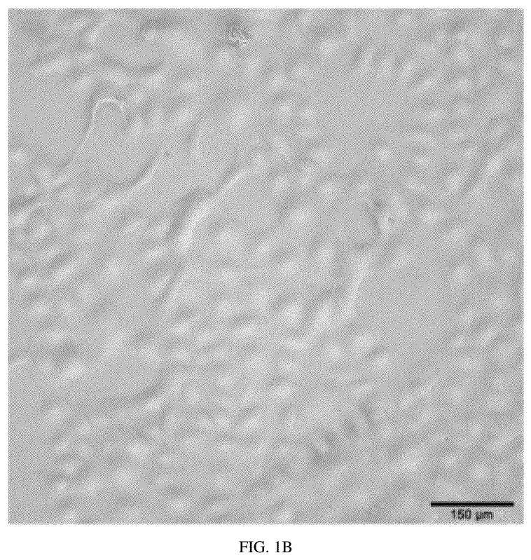

The fluid containing follicle envelope is filtered by a 40 micrometer (μm) cell sieve (Sigma), centrifuged at 1000 revolutions per minute (rpm) for 7 minutes (min) and the supernatant is discarded; the liquid is added with 5 mL of MEM (Sigma) containing 10% fetal bovine serum and 1% Penicilllin-Streptomycin Solution to resuspend the cells, the cells are transferred to a 25 cm² culture flask, and the culture flask is put into a humidified incubator containing 5% carbon dioxide at 18±0.2° C. for culture. FIG. 1A and FIG. 1B show a growth state of ovarian granulosa cells of Oncorhynchus mykiss under different adherence time, where FIG. 1A is a morphological diagram of the ovarian granulosa cells of Oncorhynchus mykiss under a microscope with a magnification of 10×, FIG. 1B is the morphological diagram of ovarian granulosa cells of Oncorhynchus mykiss under a microscope with a magnification of 20×. As shown in FIG. 1A and FIG. 1B, the ovarian granulosa cells of Oncorhynchus mykiss grow in a monolayer adherence after 24 h of separation in vitro, with a spindle or irregular polygon shape, oval nucleuses in the center, and a number of protrusions protruding from the cytoplasm and most growing in a radial or vortex shape.

2. Passage Culture of the Ovarian Granulosa Cells of Oncorhynchus mykiss

When the primary granulosa cells are adherent to the wall of the culture flask and the monolayer cell area reaches 80%, the medium in the original culture flask is suck out, and the original culture flask is added with PBS solution for gently rinsing and the solution is suck out; the original culture flask is added with 2 mL of 0.25% trypsin digestive solution (Gibco) to digest for 70 seconds (s), and the trypsin is suck out; the original culture flask is then added with 2 mL of complete MEM containing serum and Penicilllin-Streptomycin Solution to stop digestion, and a pipette gun is used to repeatedly blow the cells for 8 times to make single cell suspension; 1 mL of single cell suspension is sucked respectively to another 25 cm² culture flasks, with a volume of each flask fixed to 5 mL for continuous culture where passaging once is carried out about every 4 days.

### 3. Cryopreservation and Resuscitation of Cells

The granulosa cells in this embodiment are put into liquid nitrogen for long-term storage every five generations from the fifth generation. Resuscitation is carried out every 30 days, and the growth of cells after resuscitation is observed under microscope. The survival rate of cells is 60%. The results show that there is no significant difference in growth state after the cells passage for more than 30 times.

4. Identification of Ovarian Granulosa Cells of Oncorhynchus mykiss
1) Identification of Ovarian Granulosa Cells of Oncorhynchus mykiss by an Immunofluorescence Method

According to the characteristics of specific expression of follicle-stimulating hormone receptor (FSHR) in granulosa cells, the granulosa cells cultured in vitro are identified by the cell immunofluorescence method. The specific operation steps are as follows:
a, sucking out the old culture medium of granulosa cells cultured for 24 h, and washing the granulosa cells with PBS for 3 times, 5 min for each time;
b, fixing by 4% paraformaldehyde for 1 h, and washing with PBS for 3 times, 5 min for each time;
c, incubating with 0.3% TritionX-100 on ice for 15 min, and washing with PBS for 3 times, 5 min for each time;
d, incubating with blocking solution (0.5% fetal bovine serum+0.3% TritonX-100) at room temperature for 2 h, and washing with PBS for 3 times, 5 min for each time;
e, adding primary antibody (anti-FSHR, 1:200), incubating overnight at 4° C., and washing with PBS for 3 times, 5 min for each time;
f, adding secondary antibody (fluorescein isothiocyanatefluorescein, FITC for short, 1:100), incubating at 4° C. for 1 h, and keeping away from light;
g, washing with PBS for 3 times, 5 min for each time, and dripping 4′,6-diamidino-2-phenylindole (DAPI) for nucleus staining for 10 min; and
h, observing the color development under a fluorescent microscope, and taking photos for retention.

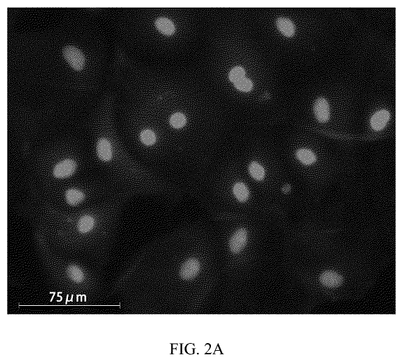

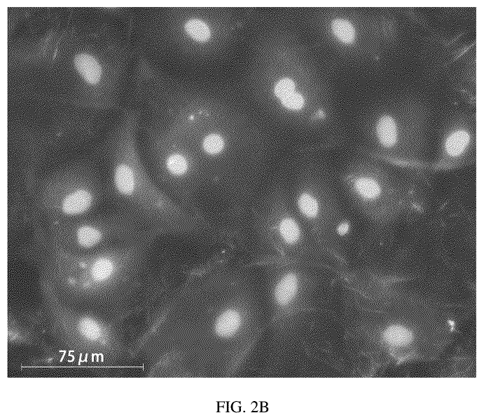

FIG. 2A-FIG. 2B show results of FSHR immunofluorescence identification of ovarian granulosa cells of Oncorhynchus mykiss, where FIG. 2A shows cells before antibody staining, FIG. 2B shows stained cells. It can be seen that the nucleuses after staining are white, which are located in cytoplasm, indicating that the cells obtained by separation are primary ovarian granulosa cells of Oncorhynchus mykiss.

### 2) Investigation on the Expression Levels of Granulosa Cell Marker Genes

Studies show that cyp19a1, fox12a and shbgb (sex hormone-binding globulinb) are all specifically expressed in ovarian granulosa cells and may be used as markers of granulosa cells. In order to further confirm that the cells separated in this embodiment are ovarian granulosa cells, the relative expression of cyp19a1, fox12a and shbgb in the cell line is investigated. To extract total RNA from cells, refer to the instructions of Simply P Total RNA Extraction Kit (Bio Flux) for specific methods. The cDNA obtained by reverse transcription is used as a template, and the gonad cell line RTG2 of Oncorhynchus mykiss is used as a control to carry out a real-time fluorescence quantitative polymerase chain reaction (real-time PCR) to detect the relative expression of granulosa cell marker genes in the cells. The reaction system uses 10 μL of SYBR Green Master Mix from Roche Company, including 5 μL of SYBR Green, 0.4 μL of upstream and downstream primers in total, 0.5 μL of cDNA template, and the balance of ddH₂O (double distilled water). The relative expression of granulosa cell marker genes is detected by 2^{-ΔΔCt} method by using BIO-RAD CFX96 TOUCH fluorometer. The PCR reaction conditions are as follows: 95° C., 10 min; 95° C., 10 s; 60° C., 30 s; a total of 40 cycles. The melting curve rises from 65° C. to 95° C., increasing by 0.5° C. per second. β-actin is used as the reference gene, and the primers are shown in Table 1.

TABLE 1

| Primers | Sequence |
|---|---|
| Oscyp19a1-qF | TGCGTCAGTGTGTCTACCATGT |
| (SEQ ID NO. 1) | |
| Oscyp19a1-qR | AGACATGTCCTCCTGGAACA |
| (SEQ ID NO. 2) | |
| Osfox12a-qF | TGTGCTGGATTTGTTTTTTGTT |
| (SEQ ID NO. 3) | |
| Osfox12a-qR | GTGTCGTGGACCATCAGGGCCA |
| (SEQ ID NO. 4) | |
| Osshbgb-qF | ACATGTGGGGGATGTTCATT |
| (SEQ ID NO. 5) | |
| Osshbgb-qR | GAGGCCATGTTACGGTTTTG |
| (SEQ ID NO. 6) | |
| Osß-actin-qF | ATCCTGACGGAGCGCGGTTACAGC |
| (SEQ ID NO. 7) | |
| Osß-actin-qR | TGCCCATCTCCTGCTCAAAGTCCA |
| (SEQ ID NO. 8) | |

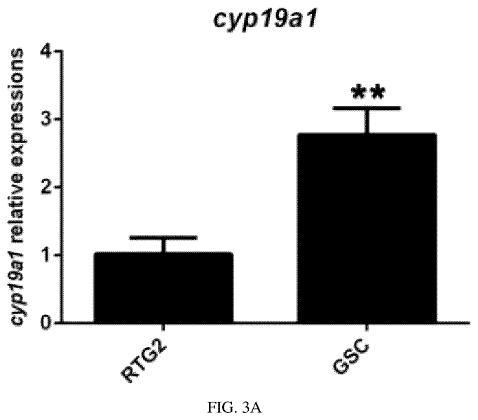

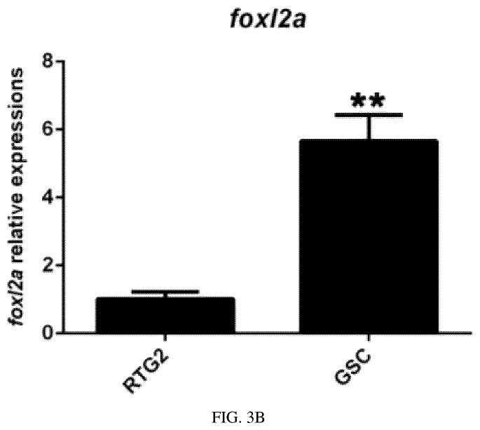

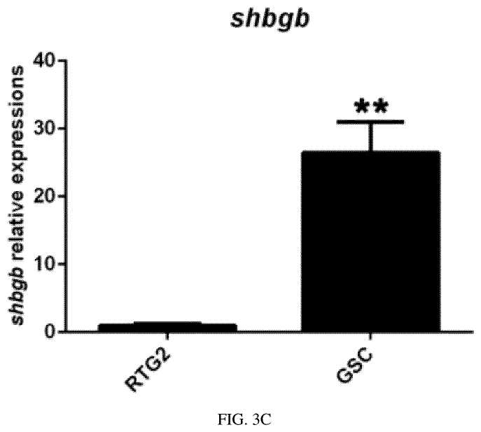

FIG. 3A, FIG. 3B and FIG. 3C show results of gene expression levels of granulosa cell marker genes cyp19a1, fox12a and shbgb, respectively, with * indicating that there is a significant difference compared with the control group (p<0.05), and ** indicating an extremely significant difference compared with the control group (P<0.01). It can be seen that compared with RTG2 cell, the cyp19a1 gene in the cells separated in this study is significantly up-regulated by 2.77 times as that in the control group. Fox12a (Forkhead transcription factor gene 2) is a member of the Forkhead transcription factor superfamily (Fox). Previous studies have found that this gene is mainly expressed in pituitary and ovarian granulosa cells and participates in the proliferation and differentiation of granulosa cells. The Fox12a gene in the granulosa cells separated in this disclosure is 26.49 times that of the control group. Similarly, the granulosa cell marker gene shbgb is 5.66 times higher than that of the control group. The above results further confirm that the cells obtained by separation are primary ovarian granulosa cells of Oncorhynchus mykiss.

### Comparative Embodiment 1

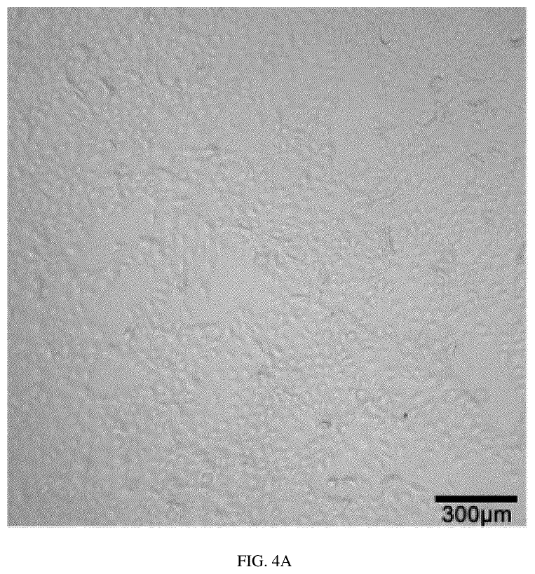

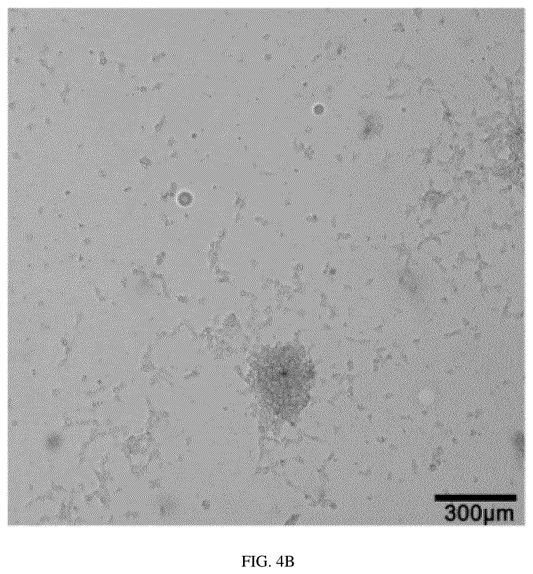

The difference between this comparative embodiment and Embodiment 1 is that in the separation and primary culture of ovarian granulosa cells of Oncorhynchus mykiss, the outer membrane of tissue is cut open with sterile tweezers, and then a single follicle is separated and transferred to complete MEM without collagenase H for incubation and digestion for 3 h. The remaining steps are the same as those in Embodiment 1. FIG. 4A and FIG. 4B are morphological diagrams of granulosa cells of Oncorhynchus mykiss, where FIG. 4A is a morphological diagram of cells incubated and digested in MEM containing collagenase H. FIG. 4B is a morphological diagram of cells incubated and digested in MEM without collagenase H. The separation results show that the primary ovarian granulosa cell body of Oncorhynchus mykiss separated in Embodiment 1 is full and the cell edge is smooth, as shown in FIG. 4A. However, the primary cell body incubated and digested with complete MEM without collagenase H in this comparative embodiment is wrinkled with blurring cell edge, as shown in FIG. 4B.

### Comparative Embodiment 2

The difference between this comparative embodiment and Embodiment 1 is that during the separation and primary culture of ovarian granulosa cells of Oncorhynchus mykiss, the cells are resuspended and transferred to 25 cm² culture flasks, and the culture flasks are put into two humidified incubators containing 5% carbon dioxide at 20° C. and 16° C. respectively for culture. The remaining steps are the same as those in Embodiment 1. The growth rate is observed, with results showing that the growth rate of Embodiment 1 cultured at 18° C. is different from that of this comparative embodiment cultured at 20° C. and 16° C. respectively. The cells cultured at 18° C. are full and grow rapidly, and it takes 3-4 days for the monolayer cell area to reach more than 80%, while the cells grow slowly at 20° C. and 16° C., and it usually takes 7-8 days for the monolayer cell area to reach 70%-80%.

The above-mentioned embodiments only describe the preferred mode of the disclosure, and do not limit the scope of the disclosure. Under the premise of not departing from the design spirit of the disclosure, various modifications and improvements made by ordinary technicians in the field to the technical scheme of the disclosure shall fall within the protection scope defined by the claims of the disclosure.

What is claimed is:

1. A construction method of an ovarian granulosa cell line of Oncorhynchus mykiss, comprising following steps:
(1) transferring a single follicle to a complete MEM containing collagenase H with a concentration of 0.4 UI/mL for digestion at 18° C. for 3-4 h; the single follicle is separated from an ovarian tissue washed by a tissue washing solution;
(2) putting a digested follicle in a culture dish containing HBSS, puncturing and incubating overnight to obtain a digestive solution containing follicle envelope, treating and placing the digestive solution into the complete MEM for resuspension and precipitation to obtain primary ovarian granulosa cells;
(3) transferring the primary ovarian granulosa cells to 18° C. for constant temperature culture, and passaging once every 4-5 days when a cell adherence density reaches over 80%; and
(4) identifying by an immunofluorescence method and a real-time fluorescence quantitative PCR method;
wherein in the step (2), components of the culture dish also comprise NaHCO₃ and 1% bovine serum albumin;
in the step (2), the treating comprises filtering the digestive solution with a 40 μm cell sieve, centrifuging at 1000 rpm for 7 min, and discarding a supernatant; components of the MEM also comprise 10% fetal bovine serum and 1% Penicilllin-Streptomycin Solution;
in the step (4), the immunofluorescence method uses a granulosa cell marker gene FSHR as a marker to carry out immunofluorescence detection in granulosa cells; the real-time fluorescence quantitative PCR method is based on expression levels of cyp19a1, fox12a and shbgd in the ovarian granulosa cells.

2. An application of the ovarian granulosa cell line obtained by the construction method according to claim 1 in separation and culture of fish granulosa cells.
